(19) United States
(12) Patent Application Publication
(10) Pub. No.: US 2013/0238030 A1
(43) Pub. Date: Sep. 12, 2013

(54) SYSTEMS AND METHODS FOR LINKING IMPLANTS AND REDUCING DEFORMITIES

(71) Applicant: Jean-Paul Steib, Strasbourg (FR)

(72) Inventor: Jean-Paul Steib, Strasbourg (FR)

(21) Appl. No.: 13/403,150

(22) Filed: Feb. 23, 2012

Related U.S. Application Data
(60) Provisional application No. 61/446,361, filed on Feb. 24, 2011.

(51) Int. Cl. A61B 17/70

(52) U.S. Cl. CPC A61B 17/7002
USPC 606/264; 606/279

(57) ABSTRACT

A system for linking bone anchors and reducing spinal deformities includes a first pair of bone screws, a first cross-link, a second pair of bone screws, a second cross-link, and a longitudinal member. The first pair of bone screws attach to opposite sides of a first vertebral level. The first cross-link includes end members that removably attach to heads of the first pair of bone screws and a first coupling feature disposed between the end members. The second pair of bone screws attach to opposite sides of a second vertebral level. The second cross-link includes end members that removably attach to heads of the first pair of bone screws and a second coupling feature disposed between the end members. The longitudinal member links the first coupling feature to the second coupling feature to enable simultaneous positioning of the first vertebral level with the second vertebral level.

### CROSS REFERENCES TO RELATED APPLICATIONS

This application claims priority to U.S. Provisional Application No. 61/446,361, filed on Feb. 24, 2010, and is incorporated herein by reference in its entirety. This application is related to co-pending U.S. application Ser. No. ______, docket number: 0137US2 entitled “APPARATUS FOR LINKING IMPLANTS AND REDUCING DEFORMITIES”.

### FIELD

The present disclosure generally relates to the field of spinal orthopedics, and more particularly to systems and methods for linking spinal implants and reducing spinal deformities.

### BACKGROUND

The spine is a flexible column formed of a plurality of bones called vertebrae. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibrocartilaginous bodies. Various spinal disorders may cause the spine to become misaligned, curved, and/or twisted or result in fractured and/or compressed vertebrae. It is often necessary to surgically correct these spinal disorders.

The spine includes seven cervical (neck) vertebrae, twelve thoracic (chest) vertebrae, five lumbar (lower back) vertebrae, and the fused vertebrae in the sacrum and coccyx that help to form the hip region. While the shapes of individual vertebrae differ among these regions, each is essentially a short hollow shaft containing the bundle of nerves known as the spinal cord. Individual nerves, such as those carrying messages to the arms or legs, enter and exit the spinal cord through gaps between vertebrae.

The spinal disks act as shock absorbers, cushioning the spine, and preventing individual bones from contacting each other. Disks also help to hold the vertebrae together. The weight of the upper body is transferred through the spine to the hips and the legs. The spine is held upright through the work of the back muscles, which are attached to the vertebrae. While the normal spine has no side-to-side curve, it does have a series of front-to-back curves, giving it a gentle “S” shape. If the proper shaping and/or curvature are not present due to scoliosis, neuromuscular disease, cerebral palsy, or other disorder, it may be necessary to straighten or adjust the spine into a proper curvature.

Generally the correct curvature is obtained by manipulating the vertebrae into their proper position and securing that position with a rigid system of screws and rods. The screws may be inserted into the pedicles of the vertebrae to act as bone anchors, and the rods may be inserted into heads of the screws. Two rods may run substantially parallel to the spine and secure the spine in the desired shape and curvature. Thus the rods, which are shaped to mimic the correct spinal curvature, force the spine into proper alignment. Bone grafts are then placed between the vertebrae and aid in fusion of the individual vertebrae together to form a correctly aligned spine.

In addition, transverse or cross-link connectors may be attached to couple one spinal rod to the other spinal rod in order to reduce torsional forces on the spinal rods and provide a more rigid construct. The connectors may include clamps or other attachment features that typically require some clearance between the spinal rod and the vertebrae and/or the heads of the screws. Various spinal features, such as the spinous process and the superior articular process, may extend between the spinal rods. These spinal features may interfere with and limit options for placement of the connectors. Thus, it may be desirable to provide a more flexible system for linking spinal rods that is less impacted by clearances and/or interference.

Spinal deformity correction procedures can require complex anatomical manipulation to restore proper curvature to the spine of a patient. Currently, some systems include shafts with handles to perform vertebral column manipulation (VCM). The shafts may be attached to the heads of the screws and a rotational force may be applied in a process referred to as “derotation.” Correction of a large, complex 3-dimensional spinal deformity can exert a high stress concentration on the bone screws and the vertebrae. High stress concentration may damage the screws, heads, and/or vertebrae the screws to breach the vertebrae. Thus, it may be desirable to distribute forces between multiple levels of the vertebrae and multiple bone screws in order to reduce the occurrence of such damage.

### SUMMARY

A system for linking bone anchors and reducing spinal deformities includes a first pair of bone screws, a first cross-link, a second pair of bone screws, a second cross-link, and a longitudinal member. The first pair of bone screws attach to opposite sides of a first vertebral level. The first cross-link includes end members that removably attach to heads of the first pair of bone screws and a first coupling feature disposed between the end members. The second pair of bone screws attach to opposite sides of a second vertebral level. The second cross-link includes end members that removably attach to heads of the first pair of bone screws and a second coupling feature disposed between the end members. The longitudinal member links the first coupling feature to the second coupling feature to enable simultaneous positioning of the first vertebral level with the second vertebral level.

In other features, each of the end members includes tracks that engage with channels on corresponding ones of the heads. Each of the cross-links includes an arcuate profile. Each coupling feature includes a projection extending from an upper surface of the cross-link. Each coupling feature includes a central disposition along each cross-link. Each coupling feature includes an aperture for receiving the longitudinal member. The longitudinal member includes a non-circular cross-section.

A method for linking bone screws and reducing spinal deformities includes steps of inserting a first pair of bone screws to opposite sides of a first vertebral level, attaching a first cross-link including end members that removably attach to heads of the first pair of bone screws and a first coupling feature disposed between the end members, inserting a second pair of bone screws to opposite sides of a second vertebral level, attaching a second cross-link including end members that removably attach to heads of the first pair of bone screws and a second coupling feature disposed between the end members, and inserting a longitudinal member that links the first coupling feature to the second coupling feature to enable simultaneous positioning of the first vertebral level with the second vertebral level.

In other features, the step of inserting the first and second cross-links includes sliding tracks that engage with channels on corresponding ones of the heads. The method includes the step of positioning at least one of the first and second vertebral levels by attaching an instrument to the coupling feature and applying an amount of force.

### BRIEF DESCRIPTION OF THE DRAWINGS

FIG. 1 is a perspective view of a system for linking implants and reducing deformities of the spine according to the principles of the present disclosure.

FIG. 2 is a perspective view of a cross-link and bone anchors of the system according to the principles of the present disclosure.

FIG. 3 is a partial perspective view of the system illustrating a longitudinal member prior to insertion into the cross-links according to the principles of the present disclosure.

FIG. 4 is a partial perspective view of the system illustrating the longitudinal member after insertion into the cross-links according to the principles of the present disclosure.

FIG. 5 is a perspective view of the cross-link and bone anchors of the system further including an instrument for derotation of the spinal column according to the principles of the present disclosure.

FIG. 6 is a perspective view of a head of an exemplary bone anchor of the system according to the principles of the present disclosure.

FIG. 7 is a partial perspective view of the cross-link, bone anchor, and spinal rod of the system according to the principles of the present disclosure.

FIG. 8 is a partial perspective view of an end of the cross-link of the system according to the principles of the present disclosure.

FIG. 9 is another partial perspective view of the end of the cross-link of the system according to the principles of the present disclosure.

### DETAILED DESCRIPTION

The systems and methods for linking implants and reducing deformities of the present disclosure include various features to enable linking of implants, such as bone anchors, pedicle screws, and the like, transversely using cross-links. The cross-links attach to the heads of the screws and eliminate the need for clearance between the spinal rods and the vertebrae. The cross-links bypass spinal features that may otherwise interfere with passage of the connector across the vertebrae. The cross-links include other features to enable linking of multiple cross-links longitudinally along the spinal column using a coupling feature and longitudinal members. The longitudinal members attach to the coupling features to link each cross-link to provide a more rigid construct. Furthermore, the cross-links include other features to enable attachment of instruments to assist with derotation of the spine using an attachment feature. The instruments may apply forces to the cross-link at the attachment feature during derotation. Because the bone anchors are linked by the cross-link and longitudinal members, stresses due to the forces may be distributed across two or more bone anchors, thus reducing the likelihood of damage to the screws, heads, and/or vertebrae.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

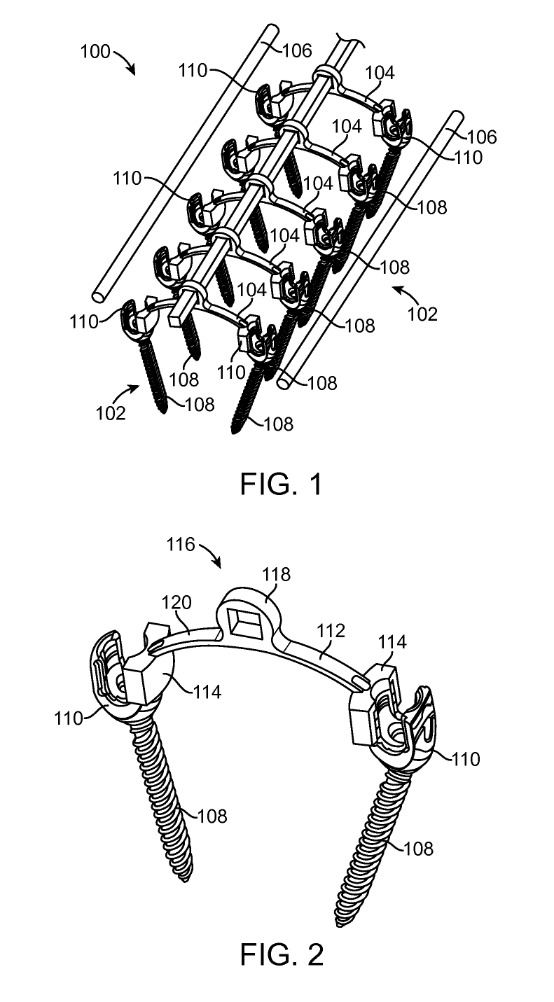

Referring now to FIG. 1, a system 100 for linking implants and reducing deformities of the spine includes bone anchors 102, cross-links 104, and spinal rods 106. A surgeon may drill and tap holes in the vertebrae to receive screws 108 of the bone anchors 102. In other examples, the screws 108 may be self-tapping. Heads 110 may be coupled to the screws 108 prior to or after securing the screws 108. The heads 110 may be configured to receive the spinal rods 106. The cross-links 104 may attach to the heads 110 to link pairs of the bone anchors 102 transversely across the spinal column. Once the spinal rods 106 have been inserted into the heads 110, a setscrew or other locking mechanism may be used to secure the rods 106. An exemplary bone anchor assembly may be found in U.S. Pub. No. 2010/0036443, entitled “Systems and Methods for Spinal Fixation”, incorporated herein by reference.

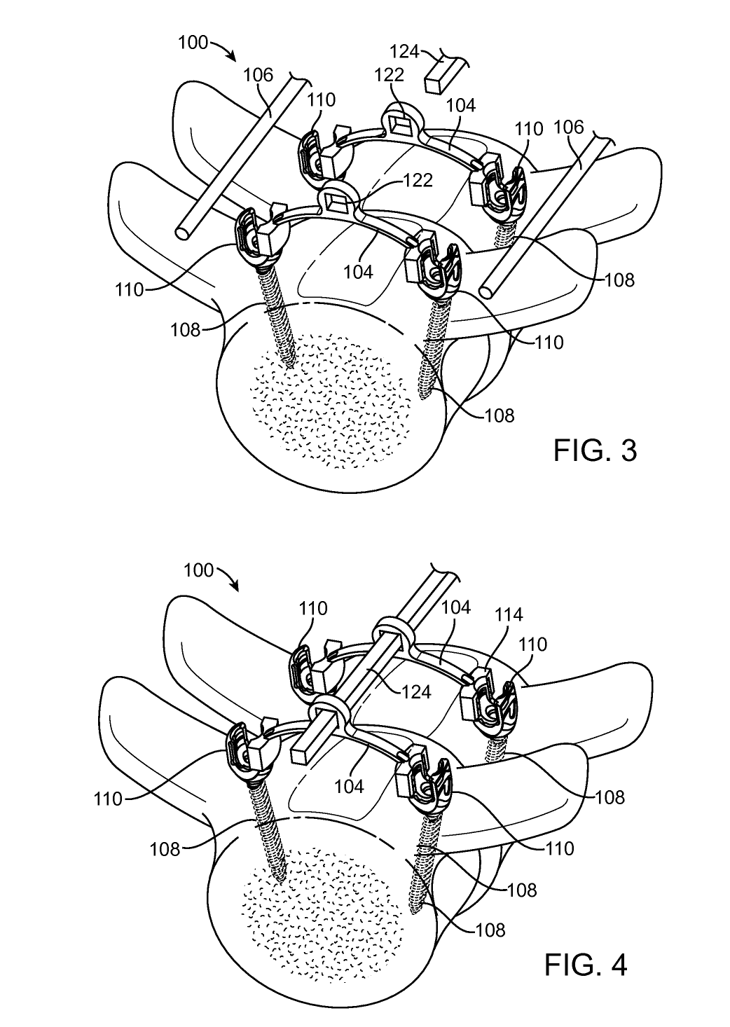

Referring now to FIG. 2, the cross-link 104 includes a transverse member 112 and end members 114 that attach to the heads 110 of the bone anchors 102. The transverse member 112 may include an arcuate profile having curvature to enable clearance of various features of the vertebrae such as the spinous process or articular processes. A coupling feature 116 may be used by the surgeon to couple multiple cross-links 104 and provide a more rigid construct. For example, the coupling feature 116 may include a projection 118 on a posterior surface 120 of the transverse member 112. The coupling feature 116 may be centrally disposed along the cross-link 104. An aperture 122 in the projection 118 may be configured to receive a longitudinal member 124 as shown in FIG. 3. For example, the aperture 122 may include a substantially rectangular cross-section corresponding to a rectangular cross-section of the longitudinal member 124. The rectangular cross-section may prevent rotation of the longitudinal member 124. Although a rectangular cross-section is shown, any non-circular cross-section may be used to prevent rotation and enable simultaneous movement of multiple levels of vertebrae. As the longitudinal member 124 is inserted through the apertures 122, multiple cross-links 104 may be secured to increase rigidity of the system 100 as shown in FIG. 4.

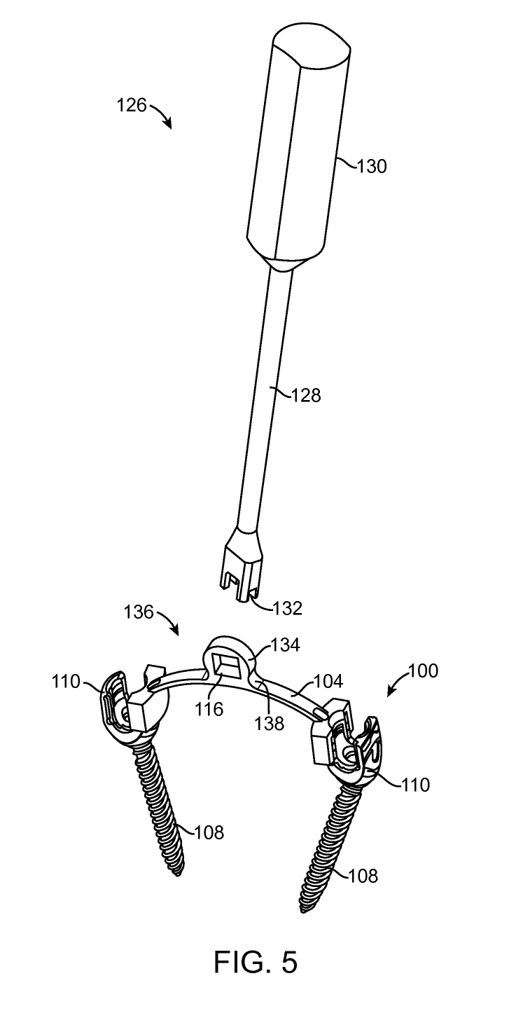

Referring now to FIG. 5, the coupling feature 116 may also include features that may be used by the surgeon to attach an instrument 126 for derotation of the spinal column. The instrument 126 may include a shaft 128 and a handle 130 which the surgeon may grip to apply force on the system 100. The shaft 128 may include an opening 132 configured to attach to the coupling feature 116. For example, the coupling feature 116 may include a contoured surface 134 that forms a substantially tear-dropped projection 136. The opening 132 may be inserted over the projection 136 and elastically expand as it passes over the projection 136 at the widest point. The opening 132 may contract at a neck 138 where the projection 136 extends form the cross-link 104. Various features may be used to attach the instrument 126 to the coupling feature 116.

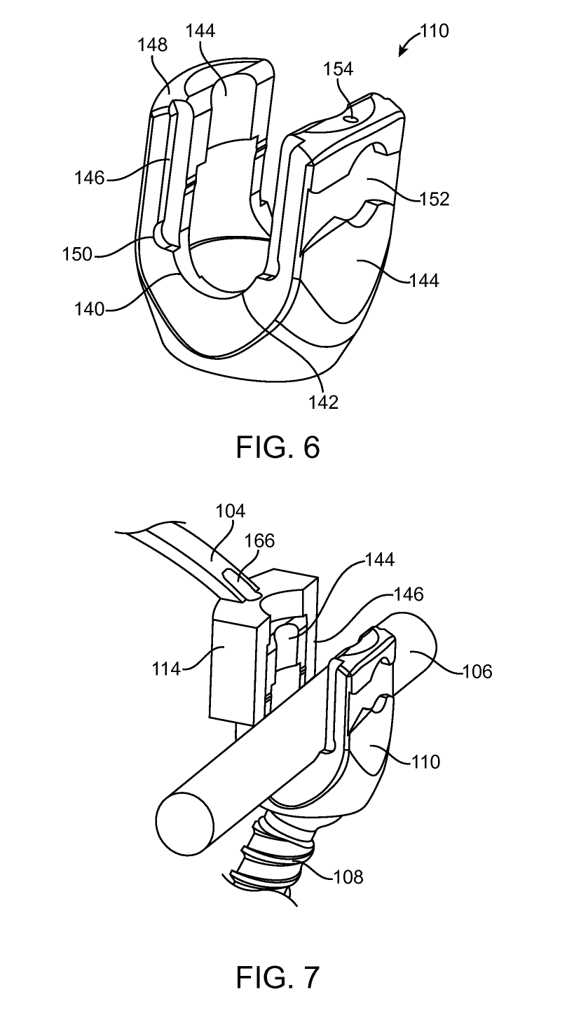

Referring now to FIG. 6, the head 110 of the bone anchor 102 may include various features that enable insertion of the spinal rod 106 and attachment of the cross-link 104. The head 110 includes a passageway 140 that is sized and configured to accommodate placement and securing of a rod. For example, the passageway 140 may include a curved distal portion 142 and two side portions 144 extending proximally therefrom to form a U-shaped portion of the head 110. The curved portion 142 may include a radius of curvature that corresponds to the diameter of the spinal rod 106. The two side portions 144 may include threads (not shown) configured to receive setscrews (not shown) that lock the spinal rods 106 within the heads 110. To secure the rod 106 inside the screw head 110, the rod 106 is placed into the passageway 140 and then the set screw is placed on top of the rod 106 and secured using threads disposed on the interior walls of the passageway 140.

The passageway 140 can be configured to include recessed edges or channels 146 that are configured to accommodate placement of the end members 114 of the cross-link 104. Each side portion 144 includes additional features for attachment of the cross-link 104. For example, the side portion 144 may include the channels 146 extending from the proximal end of the head 110 towards the curved portion 142 of the passageway 140. A proximal end of the channel 146 may be tapered towards a top surface 148 of the head 110. The taper may ease insertion of the cross-link 104 as described in detail below. The distal end of the channel 146 may include an attachment feature 150, such as a pocket, dimple, or recess that provides a snap-fit attachment of the cross-link 104. Other features may include a recessed portion 152 for attachment of various other instruments such as rod reducers and adjustment instruments for seating the spinal rod 106 within the head 110.

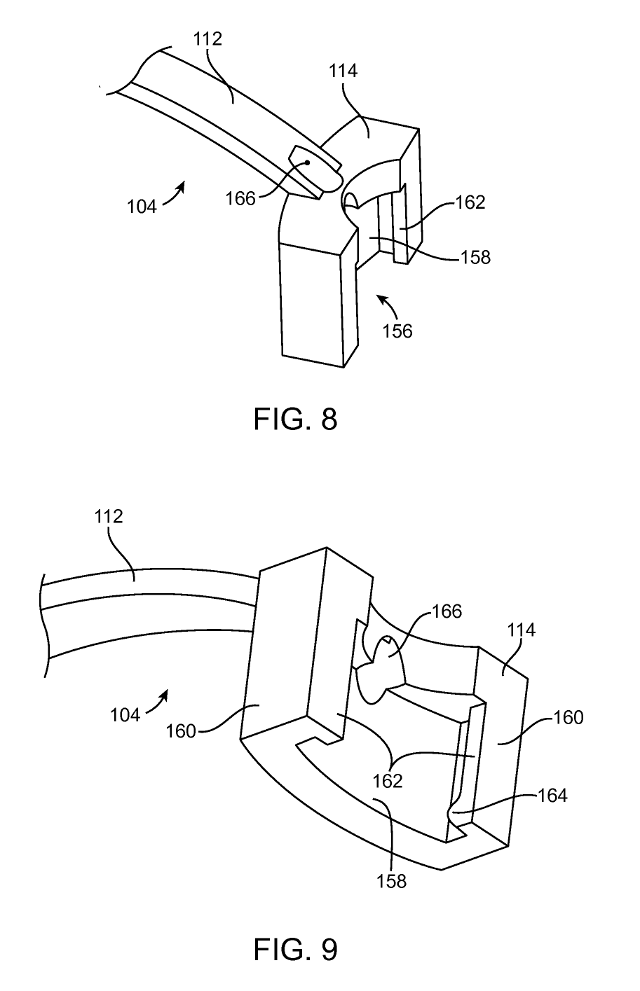

Referring now to FIGS. 7-9, the cross-link 104 includes features that may provide sliding engagement with the channels 146 on the head 110 and secure attachment to the attachment feature 150. In FIG. 8, the transverse member 112 terminates in the end member 114. The end member 114 includes a cavity 156 configured to engage with one of the side portions 144 of the head 110. For example, the cavity 156 may include a contoured portion 158 that corresponds with curvature of the side portion 144 of the head 110. Side walls 160 of the end member 114 wrap around the channels 146 on the head 110 and further include protrusions or tracks 162 that slidably engage within the channels 146. A locking feature 164 of the track 162, such as a projection or bump stop, may be configured to couple with the attachment feature 150 on the head 110. The locking feature 164 may provide permanent and/or temporary attachment of the cross-link 104 to the heads 110. In other examples, the transverse member 112 may include an aperture 166 that aligns with a threaded aperture 154 in the head 110. The aperture 166 may communicate with the posterior surface 120, passing through the end member 114, and communicate with the interior contoured portion 158. A set screw (not shown) inserted through the aperture 166 may secure the transverse member 112 to the head 110 via the threaded aperture 154.

Once the cross-links 104 are attached to the bone anchors 102, the instrument 126 may be attached to the coupling feature 116. The surgeon may begin to apply force via the instrument 126 to the system 100 to properly align the vertebrae. Because the instrument 126 attaches to the cross-links 104, any stresses due to the applied force may be distributed across the cross-link 104 to the attached bone anchors 102. In addition, when a longitudinal member 124 is also utilized, the stresses may further be distributed to bone anchors of adjacent vertebrae. Thus, any stresses that would ordinarily be experienced by a single bone anchor may be reduced by distribution of the stress among two or more bone anchors.

Furthermore, because the cross-links 104 are attached to the side portions 144 of the heads 110, the spinal rods 106 may be inserted after the surgeon has positioned the vertebrae for the correct curvature. When the spinal rods 106 are inserted prior to the derotation process, the forces on the bone anchors 102 may cause elements of the system 100 and the vertebrae to pivot about the spinal rods. Thus, without the spinal rods 106, the system 100 and vertebrae may be positioned more freely than when the spinal rods 106 are attached.

Example embodiments of the methods and systems of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

The invention claimed is:

1. A system for linking bone anchors and reducing spinal deformities, comprising:
a first pair of bone screws for attachment to opposite sides of a first vertebral level;
a first cross-link including end members that removably attach to heads of the first pair of bone screws and a first coupling feature disposed between the end members;
a second pair of bone screws for attachment to opposite sides of a second vertebral level;
a second cross-link including end members that removably attach to heads of the first pair of bone screws and a second coupling feature disposed between the end members; and
a longitudinal member that links the first coupling feature to the second coupling feature to enable simultaneous positioning of the first vertebral level with the second vertebral level.

2. The system of claim 1, wherein each of the end members includes tracks that engage with channels on corresponding ones of the heads.

3. The system of claim 1, wherein each of the cross-links includes an arcuate profile.

4. The system of claim 1, wherein each coupling feature includes a projection extending from an upper surface of the cross-link.

5. The system of claim 1, wherein each coupling feature includes a central disposition along each cross-link.

6. The system of claim 1, wherein each coupling feature includes an aperture for receiving the longitudinal member.

7. The system of claim 1, wherein the longitudinal member includes a non-circular cross-section.

8. A method for linking bone screws and reducing spinal deformities, comprising:
inserting a first pair of bone screws to opposite sides of a first vertebral level;
attaching a first cross-link including end members that removably attach to heads of the first pair of bone screws and a first coupling feature disposed between the end members;
inserting a second pair of bone screws to opposite sides of a second vertebral level;
attaching a second cross-link including end members that removably attach to heads of the first pair of bone screws and a second coupling feature disposed between the end members; and
inserting a longitudinal member that links the first coupling feature to the second coupling feature to enable simultaneous positioning of the first vertebral level with the second vertebral level.

9. The method of claim 8, wherein inserting the first and second cross-links includes sliding tracks that engage with channels on corresponding ones of the heads.

10. The method of claim 8, further comprising positioning at least one of the first and second vertebral levels by attaching an instrument the coupling feature and applying an amount of force.
